(19) United States
(12) Patent Application Publication
(10) Pub. No.: US 2026/0151017 A1
(43) Pub. Date: Jun. 4, 2026

(54) ENDOSCOPIC DIAGNOSIS ASSISTANCE METHOD, INFERENCE MODEL, ENDOSCOPIC IMAGE PROCESSING DEVICE, ENDOSCOPIC IMAGE PROCESSING SYSTEM, AND ENDOSCOPIC IMAGE PROCESSING PROGRAM

(71) Applicant: OLYMPUS MEDICAL SYSTEMS CORP., Tokyo (JP)

(72) Inventor: Akihiro KUBOTA, Kokubunji-shi (JP)

(73) Assignee: OLYMPUS MEDICAL SYSTEMS CORP., Tokyo (JP)

(21) Appl. No.: 19/462,258

(22) Filed: Jan. 28, 2026

Related U.S. Application Data
(63) Continuation of application No. PCT/JP2023/028220, filed on Aug. 2, 2023.

(51) Int. Cl. A61B 1/00; A61B 1/005; A61B 1/31; A61B 90/00; G06T 7/00; G06T 7/20

(52) U.S. Cl. CPC A61B 1/000096; A61B 1/000094; A61B 1/00045; A61B 1/00055; A61B 1/00097; A61B 1/31; A61B 90/08; G06T 7/0012; G06T 7/20; A61B 1/005; A61B 2090/0807; A61B 2560/0462; G06T 2207/10068; G06T 2207/20081; G06T 2207/30028; G06T 2207/30096

(57) ABSTRACT

An endoscopic diagnosis assistance method includes: detecting an acquisition position that is a position within a lumen whose image is acquired by an imaging portion provided at a distal end portion of an endoscope; detecting a speed at which the distal end portion of the endoscope is advanced into or retracted from the lumen; and determining, based on an inference model, whether or not the speed of the distal end portion of the endoscope passing through an attention region capable of being determined under a predetermined condition is within a predetermined speed range for the attention region relative to the detected acquisition position, wherein the inference model was obtained by machine learning using: previously captured image frames captured in multiple cases; and a result of determining the attention region for the previously captured image frames to annotate an examination speed appropriate for examining the attention region, as training data.

### CROSS-REFERENCE TO RELATED APPLICATION

This application is a continuation under 35 U.S.C. § 365 (c) of PCT Patent Application No. PCT/JP2023/028220, filed on Aug. 2, 2023, the entire content of which is hereby incorporated by this reference.

### FIELD

The present disclosure relates to an endoscopic diagnosis assistance method, an inference model, an endoscopic image processing device, an endoscopic image processing system, and an endoscopic image processing program.

### BACKGROUND

Conventionally, endoscopes have been widely used in medical and industrial fields. For example, in the medical field, a practitioner can view an endoscopic image of the inside of a subject displayed on a display device, identify a lesion, and perform treatment on the lesion using a treatment tool.

In recent years, computer-aided detection/diagnosis (CAD), which indicates candidate positions of lesions or displays differential information about endoscopic images to suppress the overlooking of lesions by a practitioner, has been developed. For example, when a lesion is detected by CAD, a diagnostic assistance function of notifying the practitioner of the presence of the lesion by presenting an emphasized display of a marker such as a frame, on the endoscopic image has been proposed.

This diagnostic assistance function is effective for confirming abnormal regions such as lesions. However, even if the diagnostic assistance function is used, there is a possibility that the practitioner may overlook an abnormal region such as a lesion, depending on the operating situation of the endoscope or the content of the endoscopic image. Accordingly, for example, Japanese Unexamined Patent Application, First Publication No. 2023-026480 (Patent Document 1) discloses a medical image processing device that attempts to prevent overlooking of lesions and the like by presenting an emphasized display of information about the lesions in accordance with real-time characteristics of a medical image.

### SUMMARY

However, the conventional diagnostic assistance functions described in Patent Document 1 and the like cannot present an emphasized display for an abnormal region such as a lesion unless the abnormal region is detected in the first place. Furthermore, in the conventional diagnostic assistance functions, before detection of an abnormal region such as a lesion, an attention region to be carefully observed is detected and overlooking of the abnormal region such as the lesion is not prevented.

The present disclosure provides an endoscopic diagnosis assistance method, an inference model, an endoscopic image processing device, an endoscopic image processing system, and an endoscopic image processing program for detecting an attention region to be carefully observed and provide a notification to a practitioner so that the region can be carefully observed to prevent overlooking of abnormal regions such as lesions.

According to an aspect of the present disclosure, there is provided an endoscopic diagnosis assistance method including: detecting an acquisition position that is a position within a lumen whose image is acquired by an imaging portion provided at a distal end portion of the endoscope; detecting a speed at which the distal end portion of the endoscope is advanced into or retracted from the lumen; and determining, based on an inference model, whether or not the speed of the distal end portion of the endoscope passing through an attention region capable of being determined under a predetermined condition is within a predetermined speed range for the attention region relative to the detected acquisition position, wherein the inference model was obtained by machine learning using: previously captured image frames captured in multiple cases; and a result of determining the attention region for the previously captured image frames to annotate an examination speed appropriate for examining the attention region, as training data.

According to an endoscopic diagnosis assistance method, an inference model, an endoscopic image processing device, an endoscopic image processing system, and an endoscopic image processing program of the present disclosure, it is possible to detect an attention region to be carefully observed and provide a notification to a practitioner so that the region can be carefully observed to prevent overlooking of abnormal regions such as lesions.

### BRIEF DESCRIPTION OF DRAWINGS

FIG. 1 is a diagram showing an endoscopic system according to a first embodiment.

FIG. 2 is an explanatory diagram of shape observation of an endoscope using an observation device.

FIG. 3 is a functional block diagram of the endoscopic system.

FIG. 4 is a functional block diagram of an attention region detection portion of the endoscopic system.

FIG. 5 is a diagram showing an example of an attention region determination table.

FIG. 6 is a diagram showing an example of a speed limit determination table.

FIG. 7 is a diagram showing an example of a composite image.

FIG. 8 is a flowchart of the endoscopic system.

FIG. 9 is a functional block diagram of an endoscopic system according to a second embodiment.

FIG. 10 is a functional block diagram of a speed determination portion in the endoscopic system.

FIG. 11 is a conceptual diagram of an inference model of the speed determination portion.

FIG. 12 is an explanatory diagram of training data.

FIG. 13 a flowchart showing a training data acquisition process.

FIG. 14 is a flowchart showing an inference model training process.

### DETAILED DESCRIPTION

### First Embodiment

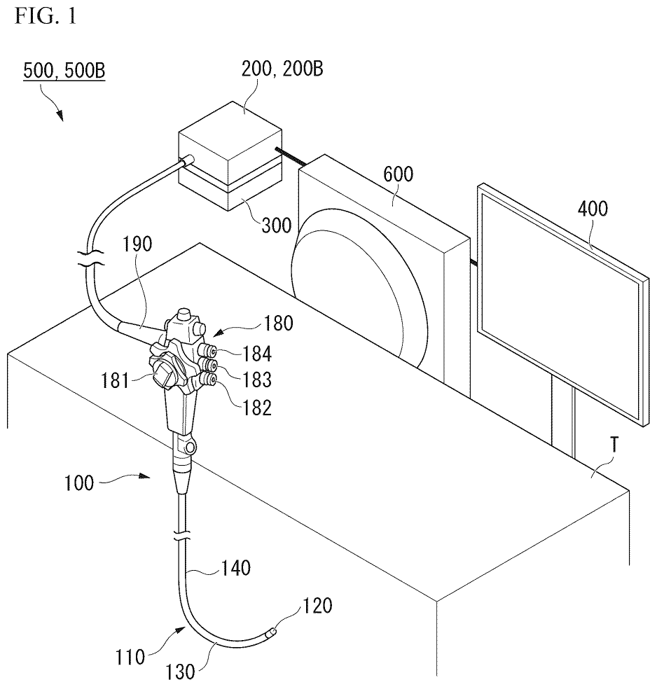

An endoscopic system 500 according to a first embodiment of the present disclosure will be described with reference to FIGS. 1 to 8.

### [Endoscopic System 500]

FIG. 1 is a diagram showing the endoscopic system 500.

The endoscopic system (endoscopic image processing system) 500 includes an endoscope 100, an image processing processor device 200, a light source device 300, a display device 400, and an observation device 600. The image processing processor device 200 and the light source device 300 may be an integrated device (an image control device).

The light source device 300 has a light source 310 such as a light-emitting diode (LED), and controls an amount of illumination light transmitted to the endoscope 100 via a light guide 161 by controlling the light source.

The display device 400 is a device that displays images generated by the image processing processor device 200, various types of information related to the endoscopic system 500, and the like. The display device 400 is, for example, a liquid crystal monitor.

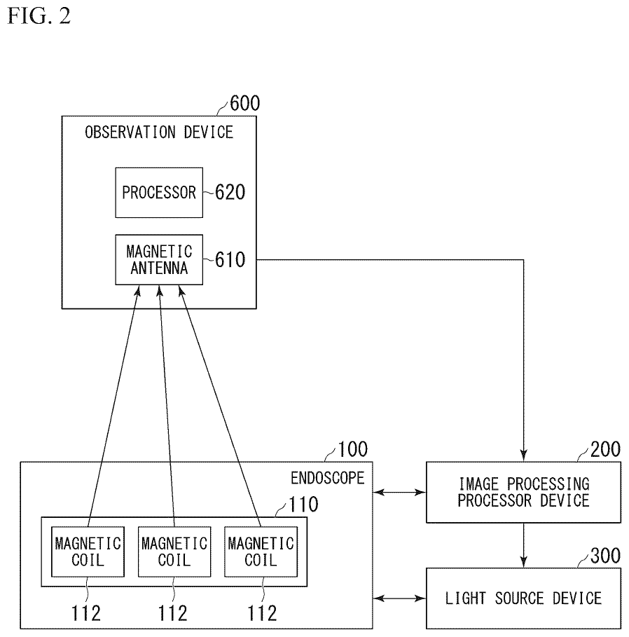

FIG. 2 is an explanatory diagram of observation of a shape of the endoscope 100 by the observation device 600.

The observation device 600 is a device for observing an insertion shape of the endoscope 100 using a magnetic field. For example, the observation device 600 receives magnetism generated from magnetic coils 112 incorporated in an insertion portion 110 of the endoscope 100 with a magnetic antenna 610. An observation result of the observation device 600 is acquired by an image processing processor device 200. The image processing processor device 200 calculates a three-dimensional position of the magnetic coil 112 from the strength of the received magnetic field using a technique called endoscope position detecting (UPD), connects the three-dimensional positions of the magnetic coil 112 with a smooth curve, and further performs graphic processing so that three-dimensional positions of the magnetic coils 112 is more easily recognized, thereby generating an image of the insertion shape of the endoscope 100. The generated image of the insertion shape of the endoscope 100 is displayed on a display device 400.

### [Endoscope 100]

The endoscope 100 is, for example, a device for observing and treating the inside of a patient lying on an operating table T. The endoscope 100 includes a long and thin insertion portion 110 to be inserted into the patient's body, a manipulation portion 180 connected to a proximal end of the insertion portion 110, and a universal cord 190 extended from the manipulation portion 180.

The insertion portion 110 includes a distal end portion 120, a bending portion 130 that can be bent freely, and a flexible tube portion 140 that is long and flexible. The distal end portion 120, the bending portion 130, and the flexible tube portion 140 are connected in that order from the distal end side. The flexible tube portion 140 is connected to the manipulation portion 180.

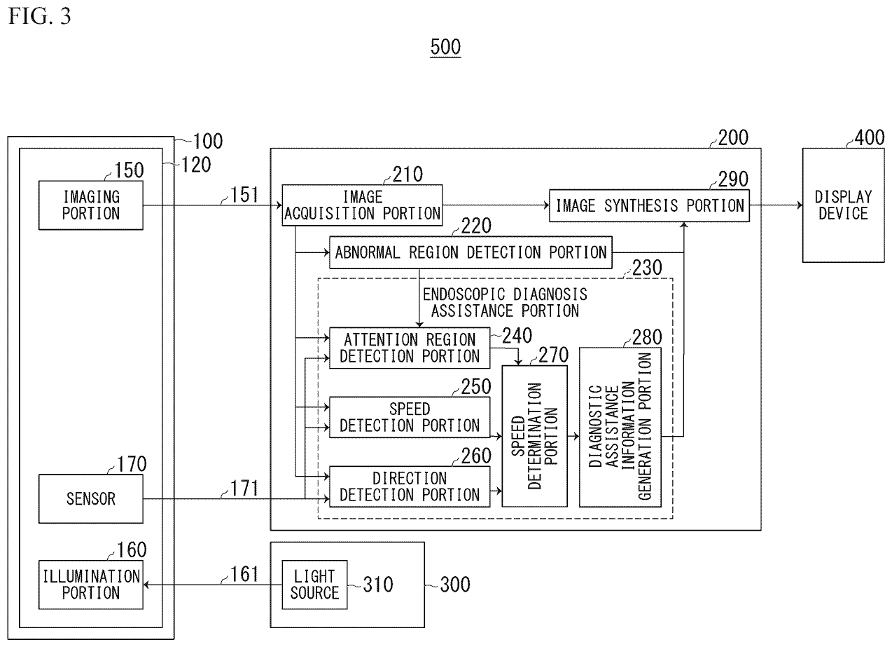

FIG. 3 is a functional block diagram of the endoscopic system 500. Hereinafter, the functional block of the endoscopic system 500 shown in FIG. 3 will be described with reference to the configuration of the endoscopic system 500 shown in FIG. 1. The distal end portion 120 has an imaging portion 150, an illumination portion 160, and a sensor 170.

The imaging portion 150 has an optical system, an imaging element configured to convert an optical signal into an electrical signal, and an analog-to-digital (AD) conversion circuit configured to convert an analog signal output by the imaging element into a digital signal. The imaging portion 150 captures an image of a subject and generates an imaging signal. The imaging signal is acquired by the image processing processor device 200 via an imaging signal cable 151.

The illumination portion 160 irradiates the subject with illumination light transmitted by the light guide 161. The light guide 161 is inserted through the insertion portion 110, the manipulation portion 180, and the universal cord 190 and connected to the light source device 300. The illumination portion 160 may have a light source such as an LED, or an optical element such as a phosphor with a wavelength conversion function.

The sensor 170 detects a position of the distal end portion 120 and a speed and direction of the distal end portion 120. The sensor 170 is, for example, an acceleration sensor, a gyro sensor, a combination of these sensors, or the like. The output of the sensor 170 is acquired by the image processing processor device 200 via a signal cable 171.

The manipulation portion 180 (see FIG. 1) receives a manipulation on the endoscope 100. The manipulation portion 180 has an ankle knob 181 configured to control the bending portion 130, an air/water supply button 182, a suction button 183, and a release button 184. Manipulations input to the air/water supply button 182, the suction button 183, and the release button 184 are acquired by the image processing processor device 200. The release button 184 is a push button configured to input a manipulation to save the captured image acquired from the imaging portion 150. The ankle knob 181 is a rotating handle that bends the bending portion 130. By bending the bending portion 130, it is easy to insert and remove the insertion portion 110.

The universal cord 190 (see FIG. 1) connects the endoscope 100 and the image processing processor device 200. The universal cord 190 is a cable through which the imaging signal cable 151, the light guide 161, the signal cable 171, and the like pass.

### [Image Processing Processor Device 200]

As shown in FIG. 3, the image processing processor device (endoscopic image processing device) 200 includes an image acquisition portion 210, an abnormal region detection portion 220, an endoscopic diagnosis assistance portion 230, and an image synthesis portion 290.

The image processing processor device 200 is a programmable computer that includes a processor such as a central processing portion (CPU), a memory, a recording portion, and the like. The functions of the image processing processor device 200 are implemented by the processor executing a program (an endoscopic image processing program or the like). At least some of the functions of the image processing processor device 200 may be implemented by a dedicated logic circuit mounted on an application-specific integrated circuit (ASIC) or a field-programmable gate array (FPGA).

The image processing processor device 200 may further include constituent elements other than a processor, a memory, and a recording portion. For example, the image processing processor device 200 may further include an image calculation portion configured to perform a part or all of image processing or an image recognition process. When the image calculation portion is further included, the image processing processor device 200 can execute the specific image processing or the image recognition processing at a high speed. The image calculation portion may be a calculator provided in a cloud server connected via the Internet.

The recording portion is a non-volatile recording medium configured to store the above-described program and data required for executing the program. The recording portion includes, for example, a writable non-volatile memory such as a read-only memory (ROM), or a flash memory, a portable medium such as a compact disc (CD-ROM), or a storage device such as a hard disk or a solid-state drive (SSD) built into a computer system. The recording portion may be a storage device provided in a cloud server connected via the Internet or the like.

The above-described program, for example, may be provided by a “computer-readable recording medium” such as a flash memory. The program may be transmitted from a computer that holds the program to the memory or recording portion via a transmission medium or by transmission waves in the transmission medium. The “transmission medium” for transmitting the program refers to a medium having a function of transmitting information. The medium having a function of transmitting information includes a network (a communication network) such as the Internet or a communication circuit (a communication line) such as a telephone circuit. Moreover, the above-described program may be a program for implementing some of the above-described functions. Furthermore, the above-described program may be a differential file (a differential program). The above-described functions may be implemented by a combination of the program already recorded on the computer and the differential program.

The image acquisition portion 210 acquires an imaging signal from the imaging portion 150 of the endoscope 100 via an imaging signal cable 151. The image acquisition portion 210 performs imaging signal processing on the imaging signal acquired from the imaging portion 150 to generate a captured image D. The imaging signal processing includes, for example, image adjustment (image construction) such as demosaicing, gain adjustment, white balance adjustment, gamma correction, noise reduction, contrast enhancement, and color change processing and the like.

The image acquisition portion 210 outputs the acquired captured image D to an image synthesis portion 290. Moreover, the image acquisition portion 210 outputs the acquired captured image D to the abnormal region detection portion 220 and the endoscopic diagnosis assistance portion 230.

The abnormal region detection portion 220 detects an abnormal region (an attention region or a region of interest) from the captured image D. The abnormal regions detected by the abnormal region detection portion 220 include the following regions.

The abnormal region detection portion 220, for example, detects a lesion as an abnormal region (region A). Detection of the lesion includes lesion detection (lesion position detection, lesion differentiation, lesion progression determination, and the like). The abnormal region detection portion 220 detects a lesion from the captured image D with a lesion detection machine learning model generated through machine learning using captured images D for training. The lesion detection machine learning model may be trained for each of conditions for a subject site and a type of used light source (a normal light source or a special light source), and a condition-specific machine learning model may be generated.

The abnormal region detection portion 220, for example, detects a region having an abnormal color tone, such as residue or bleeding, as an abnormal region. Residue contains a large number of ocher components. Bleeding contains a large number of red components. Based on such characteristics, the abnormal region detection portion 220 detects a region having an abnormal color tone as an abnormal region.

The abnormal region detection portion 220 detects a region where insufflation is insufficient as an abnormal region. The abnormal region detection portion 220 may detect a region where insufflation is insufficient from the captured image D with a machine learning model that has been trained in advance so that it is possible to detect a region with insufficient insufflation based on a width, wrinkles, and the like of the lumen in the captured image D.

The abnormal region detection portion 220, for example, detects a peristaltic region as an abnormal region. The abnormal region detection portion 220 determines a region moving at a speed equal to or greater than a predetermined speed as a peristaltic region, for example, based on the captured image D and information from the sensor 170.

When the abnormal region detection portion 220 detects an abnormal region, information about the abnormal region (such as the position and contents of the abnormal region) is output to the attention region detection portion 240 and the image synthesis portion 290.

The endoscopic diagnosis assistance portion 230 detects an attention region to be carefully observed from the captured image D and notifies the practitioner to carefully observe the attention region. Moreover, the endoscopic diagnosis assistance portion 230 also generates diagnostic assistance information about the attention region. Here, the “attention region” refers to a region that can be determined according to predetermined conditions and includes at least one of an abnormal region (region A) such as a lesion, and a structural region (region B) to be carefully observed that is determined by a segmented structure (site) within the lumen. The structural region (region B) to be carefully observed that is determined by the segmented structure (site) within the lumen includes, for example, a region (region B1) where an abnormal region such as a lesion is likely to occur, a region (region B2) having a complicated structure such as a bending portion where overlooking is likely to occur, a region (region B3) where there are many portions to be observed, and the like.

The endoscopic diagnosis assistance portion 230 may be a device separate from the image processing processor device 200 (hereinafter also referred to as an “endoscopic diagnosis assistance device”). The endoscopic diagnosis assistance device may be a computing device provided on a cloud server connected via the Internet.

As shown in FIG. 3, the endoscopic diagnosis assistance portion 230 includes an attention region detection portion 240, a speed detection portion 250, a direction detection portion 260, a speed determination portion 270, and a diagnostic assistance information generation portion 280.

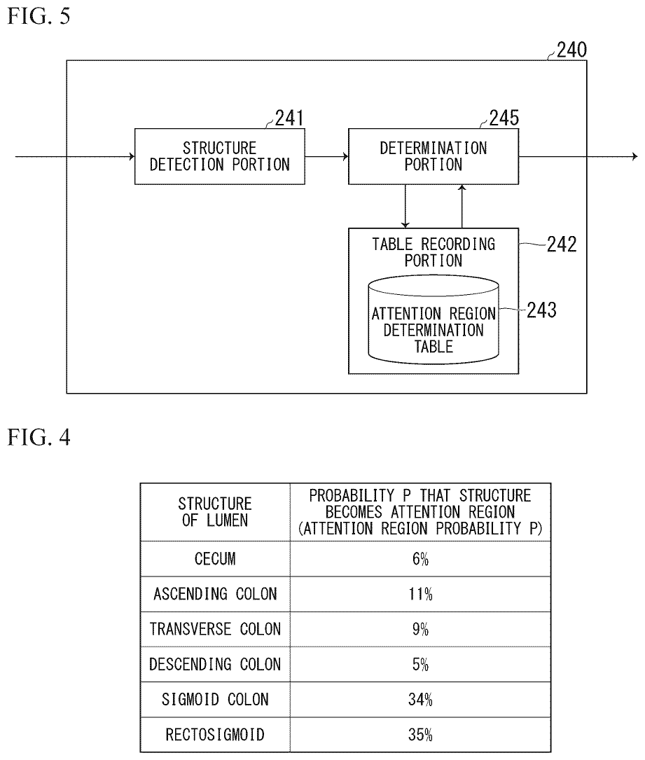

FIG. 4 is a functional block diagram of the attention region detection portion 240.

The attention region detection portion 240 acquires the detection result of an abnormal region (region A) such as a lesion from the abnormal region detection portion 220 and detects the abnormal region (region A) as an attention region. Moreover, the attention region detection portion 240 detects, as an attention region, a structural region (region B) to be carefully observed, which is determined by the segmented structure (site) within the lumen. The attention region detection portion 240 includes a structure detection portion 241, a table recording portion 242, and a determination portion 245. The structure detection portion 241, the table recording portion 242, and the determination portion 245 detect the structural region (region B) to be carefully observed.

The structure detection portion 241 detects a position within the lumen (hereinafter also referred to as an “acquisition position”) where a distal end portion 120 of the endoscope 100 acquires the captured image D. When the insertion portion 110 of the endoscope 100 is inserted into the large intestine, the structure detection portion 241 identifies the acquisition position of the captured image D based on “segmented structures (sites) in the large intestine,” such as the cecum, ascending colon, transverse colon, descending colon, sigmoid colon, and rectosigmoid. When the insertion portion 110 of the endoscope 100 is inserted into the stomach, the structure detection portion 241 identifies the acquisition position of the captured image D based on “segmented structures (sites) in the stomach,” such as the pharynx, esophagus, and stomach interior. Also, the acquisition position identified by the structure detection portion 241 is not limited to “segmented structures (sites) in the lumen,” but may instead be coordinate values or the like.

The structure detection portion 241 may (1) detect the acquisition position of the captured image D based on the captured image D, (2) detect the acquisition position of the captured image D based on the output of the sensor 170, or (3) detect the acquisition position of the captured image D based on the insertion shape of the endoscope 100 detected by the observation device 600. The detection methods (1) to (3) will be described below.

(1) The structure detection portion 241 may identify a structure of the lumen included in the captured image D by pattern matching. For example, the structure detection portion 241 compares the captured image D with pre-recorded images of respective sites and identifies a structure of the lumen included in the captured image D based on a similarity level for each of the pre-recorded images of the respective sites. The structure detection portion 241 may identify a structure (site) of the lumen included in the captured image D by inference with an inference model. For example, the inference model is obtained through machine learning using the pre-recorded images of the respective sites as training data.

(2) The structure detection portion 241 may identify a structure of the lumen included in the captured image D based on the output of the sensor 170 (such as a speed, direction, or posture of the distal end portion 120).

(3) The structure detection portion 241 may identify a structure of the lumen included in the captured image D based on an insertion shape of the endoscope 100 detected by the observation device 600. Specifically, the structure detection portion 241 detects a position of the distal end portion 120 of the endoscope 100 based on a three-dimensional shape of the insertion portion 110 detected by the observation device 600, and identifies a structure of the lumen included in the captured image D.

The structure detection portion 241 may identify a structure of the lumen included in the captured image D by combining the detection methods (1) to (3) described above.

The structure detection portion 241 transmits an acquisition position within the lumen (a segmented structure within the lumen) at which the distal end portion 120 of the endoscope 100 acquires the captured image D to the determination portion 245.

The determination portion 245 determines an attention region level of the acquisition position within the lumen (the segmented structure within the lumen) acquired from the structure detection portion 241 based on predetermined conditions. Specifically, the determination portion 245 determines whether the acquisition position within the lumen (the segmented structure within the lumen) acquired from the structure detection portion 241 belongs to a low-speed region L1, a normal-speed region L2, or a non-determination region L3. The low-speed region L1 is a region where it is necessary to move the distal end portion 120 of the endoscope 100 at a low speed as an attention region. The normal-speed region L2 is a region where the distal end portion 120 of the endoscope 100 can be operated at a normal speed or without speed limitation as a region other than the attention region. The non-determination region L3 is, for example, a region through which the insertion portion 110 of the endoscope 100 passes when inserted into the lumen, and is a region where it is unnecessary to determine whether it is an attention region. A condition for determining the attention region level can be changed by a user. The determination portion 245 transmits the structure (site) of the lumen included in the captured image D and the attention region level to the speed determination portion 270.

The determination portion 245 may further determine the attention region level in more detail based on an attention region determination table 243 recorded in the table recording portion 242. The table recording portion 242 is a part of the aforementioned recording portion and is a nonvolatile recording medium. The table recording portion 242 records the attention region determination table 243.

FIG. 5 shows an example of the attention region determination table 243.

The attention region determination table 243 is data obtained by associating a structure of the lumen with a probability P (%) that the structure becomes an attention region (an attention region probability). Here, the “attention region probability P” is, for example, a probability (probability P1) that an abnormal region such as a lesion is likely to occur, a probability (probability P2) that overlooking is likely to occur because a structure such as a bending portion is a complicated region, or a probability (probability P3) that there are many portions to be observed. The attention region probability P may be a combination of two or more of the probabilities P1 to P3 described above.

Each of P1, P2, and P3 may be 100%.

The determination portion 245 determines an “attention region probability P (%)” corresponding to the structure of the lumen included in the captured image D acquired from the structure detection portion 241 with reference to the attention region determination table 243. The determination portion 245 transmits the structure (site) of the lumen included in the captured image D and the probability P that the structure is an attention region to the speed determination portion 270.

When the determination portion 245 acquires a detection result of an abnormal region (region A) such as a lesion from the abnormal region detection portion 220, the determination portion 245 sets the attention region probability P that the structure is an attention region to 100%.

The speed detection portion 250 detects a speed at which the distal end portion 120 of the endoscope 100 is advanceable into and retractable from the lumen. The speed detection portion 250 may (1) detect the speed of the distal end portion 120 based on an output of the sensor 170, (2) detect the speed of the distal end portion 120 based on the captured image D, or (3) detect the speed of the distal end portion 120 based on a positional change in the insertion shape of the endoscope 100 detected by the observation device 600. The detection methods (1) to (3) will be described below.

The speed detection portion 250 may detect the speed at which the distal end portion 120 of the endoscope 100 is advanceable into and retractable from the lumen based on an output of the sensor 170. Specifically, the speed detection portion 250 calculates the speed at which the distal end portion 120 is advanceable into and retractable from the lumen from an output of the sensor 170 (such as an acceleration sensor, a gyro sensor, or the like) mounted on the distal end portion 120.

The speed detection portion 250 may detect the speed at which the distal end portion 120 of the endoscope 100 is advanceable into and retractable from the lumen based on the captured image D. Specifically, the speed detection portion 250 detects the speed from the movement (optical flow) of a physical object between frames (between image frames) of the captured image D. For example, the speed detection portion 250 can detect the speed at which the distal end portion 120 is advanceable into and retractable from the lumen if it is detected how a feature of the observation site (for example, vascular pattern or thickness) changes between frames of the captured image D.

The speed detection portion 250 can calculate the speed of the distal end portion 120 based on an angle of view (determined by an imaging element size or an optical system) from an amount of movement of a specific feature point such as a blood vessel between frames.

The speed detection portion 250 may detect the speed of the distal end portion 120 by combining the detection methods (1) to (3) described above.

The detected speed of the distal end portion 120 of the endoscope 100 is acquired by the speed determination portion 270.

The direction detection portion 260 detects an advancement/retraction direction (an insertion direction and a withdrawal direction) in which the distal end portion 120 of the endoscope 100 is advanceable into and retractable from the lumen. The direction detection portion 260 may (1) detect the advancement/retraction direction of the distal end portion 120 based on an output of the sensor 170, (2) detect the advancement/retraction direction of the distal end portion 120 based on the captured image D, or (3) detect the advancement/retraction direction of the distal end portion 120 based on a positional change in the insertion shape of the endoscope 100 detected by the observation device 600. The detection methods (1) to (3) will be described below.

The direction detection portion 260 may detect the advancement/retraction direction of the distal end portion 120 of the endoscope 100 based on an output of the sensor 170. Specifically, the speed detection portion 250 detects the advancement/retraction direction of the distal end portion 120 from an output of the sensor 170 (such as an acceleration sensor, a gyro sensor, or the like) mounted on the distal end portion 120.

The direction detection portion 260 may detect the advancement/retraction direction of the distal end portion 120 of the endoscope 100 based on the captured image D. Specifically, the speed detection portion 250 detects the direction from a log (history) of the observed structures (sites) in the lumen. For example, when the order of observed gastric sites is pharynx→esophagus→stomach, the direction detection portion 260 detects that the advancement/retraction direction of the distal end portion 120 is the “insertion direction.” When the order of observed gastric sites is stomach esophagus, the direction detection portion 260 detects that the advancement/retraction direction of the distal end portion 120 is the “withdrawal direction.”

The direction detection portion 260 may detect the advancement/retraction direction of the distal end portion 120 of the endoscope 100 based on a positional change in the insertion shape of the endoscope 100 detected by the observation device 600. Specifically, the structure detection portion 241 detects a positional change of the distal end portion 120 of the endoscope 100 based on a three-dimensional shape of the insertion portion 110 detected by the observation device 600, and detects the advancement/retraction direction of the distal end portion 120.

The direction detection portion 260 may detect the advancement/retraction direction of the distal end portion 120 by combining the above-described detection methods (1) to (3).

The detected advancement/retraction direction of the distal end portion 120 of the endoscope 100 is acquired by the speed determination portion 270.

The speed determination portion 270 determines whether or not the speed of the distal end portion 120 of the endoscope 100 passing through an attention region of the lumen is within an appropriate observation speed range, based on detection results of the attention region detection portion 240, the speed detection portion 250, and the direction detection portion 260.

The speed determination portion 270 selects the appropriate observation speed range corresponding to the attention region level (the low-speed region L1, the normal-speed region L2, or the non-determination region L3). For example, the speed of the appropriate observation speed range corresponding to the low-speed region L1 is lower than that of the appropriate observation speed range corresponding to the normal-speed region L2. The appropriate observation speed range corresponding to the non-determination region L3 is unset.

When the attention region detection portion 240 determines an attention region probability P, the speed determination portion 270 determines an upper-limit speed Vₘₐₓ of the appropriate observation speed range according to, for example, Eq. (1). In Eq. (1), P denotes the attention region probability P (%), NVₘₐₓ denotes an upper-limit speed of a range of a speed appropriate for observation in a normal region that is not an attention region, and a denotes an arbitrary coefficient.

$$\begin{matrix}[ Math . 1 ] & \\ {V}_{max} = α \times ( 1 - \frac{P}{100} ) \times {NV}_{max} & ( Eq . ( 1 ) )\end{matrix}$$

The upper-limit speed Vₘₐₓ of the appropriate observation speed range determined based on Eq. (1) decreases as the attention region probability P increases.

The upper-limit speed NVₘₐₓ may be set to a different speed for each lumen structure. For example, the speed determination portion 270 may set a lower upper-limit speed NVₘₐₓ for a lumen structure in which insertion of the endoscope 100 is difficult, regardless of the presence or absence of an attention region.

The speed determination portion 270 may adjust the coefficient α according to the practitioner's skill level, thereby adjusting the upper-limit speed Vₘₐₓ of the appropriate observation speed range. For example, when a practitioner with a low skill level manipulates the endoscope 100, the speed determination portion 270 may adjust the coefficient α so that the upper-limit speed Vₘₐₓ becomes lower than when a practitioner with a high skill level manipulates the endoscope 100.

The speed determination portion 270 may also decide the upper-limit speed Vₘₐₓ of the appropriate observation speed range according to, for example, Eq. (2). In Eq. (2), P denotes the attention region probability P (%), V1 and V2 denote predetermined speeds (V1>V2), Pₜₕ denotes a threshold value of the attention region probability P, and a denotes an arbitrary coefficient.

$$\begin{matrix}[ Math . 2 ] & \\ {V}_{max} = \{ \begin{matrix}α \times V 1 & ( P < {P}_{th} ) \\ α \times V 2 & ( P \geq {P}_{th} )\end{matrix} & ( Eq . ( 2 ) )\end{matrix}$$

The upper-limit speed Vₘₐₓ of the appropriate observation speed range decided based on Eq. (2) is lower when the attention region probability P is equal to or greater than the threshold Pₜₕ, compared with when the attention region probability P is less than the threshold Pₜₕ. In other words, according to Eq. (2), when the attention region probability P is equal to or greater than the threshold Pₜₕ, an attention region is detected. Also, the number of thresholds for the attention region probability P may be two or more.

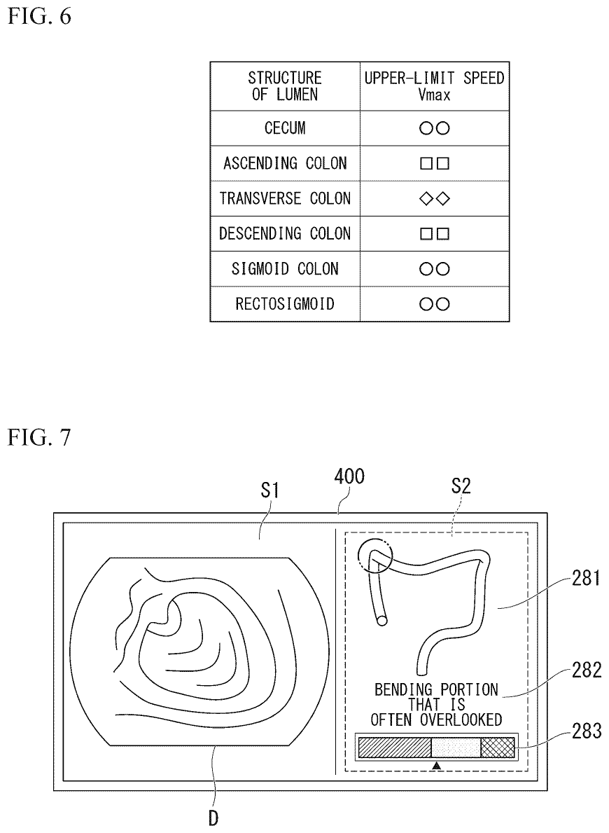

FIG. 6 is a diagram showing an example of an upper-limit speed determination table.

The speed determination portion 270, for example, may decide the upper-limit speed Vₘₐₓ of the appropriate observation speed range from a type of lumen structure, based on an upper-limit speed determination table such as shown in FIG. 6.

The speed determination portion 270 determines whether the speed of the distal end portion 120 of the endoscope 100 passing through the attention region of the lumen is within the appropriate observation speed range (including the upper-limit speed Vₘₐₓ), based on the appropriate observation speed range decided by the above-described methods. When the speed of the distal end portion 120 of the endoscope 100 is outside the appropriate observation speed range, the speed determination portion 270 notifies the diagnostic assistance information generation portion 280 of that fact.

FIG. 7 is a diagram showing an example of a composite image S1.

When the speed of the distal end portion 120 of the endoscope 100 passing through an attention region is outside the appropriate observation speed range, the diagnostic assistance information generation portion 280 generates diagnostic assistance information for the attention region. The diagnostic assistance information is information displayed in a diagnostic assistance image S2, which is a part of the composite image S1 shown in FIG. 7.

The diagnostic assistance information includes endoscope position information 281, alert information 282, and speed information 283.

The endoscope position information 281 is information indicating the position of the distal end portion 120 of the endoscope 100. As shown in FIG. 7, the endoscope position information 281 may be displayed as a diagram visualizing the position of the distal end portion 120 in the lumen.

The alert information 282 is information for warning that the distal end portion 120 of the endoscope 100 is located in an attention region. As shown in FIG. 7, the alert information 282 may be displayed as text, or may be provided through a sound notification.

The speed information 283 is information indicating the speed of the distal end portion 120 of the endoscope 100 and the appropriate observation speed range. As shown in FIG. 7, the speed information 283 may be displayed as visualized speed information by a speed meter showing the speed of the distal end portion 120 of the endoscope 100 and the appropriate observation speed range.

Also, the endoscopic diagnosis assistance portion 230 may not have the diagnostic assistance information generation portion 280. In the case where the endoscopic diagnosis assistance portion 230 does not include the diagnostic assistance information generation portion 280, when the speed determination portion 270 detects that the speed of the distal end portion 120 of the endoscope 100 is outside the appropriate observation speed range, a notification is provided to the practitioner, for example, by sound or the like. That is, a means by which the endoscopic diagnosis assistance portion 230 provides a notification to the practitioner is not limited to a display, but may also be a sound, vibrations, or the like.

The image synthesis portion 290 generates a composite image S1 including the captured image D, information about an abnormal region, and diagnostic assistance information, as shown in FIG. 7.

When the image synthesis portion 290 acquires information about an abnormal region from the abnormal region detection portion 220, the image synthesis portion 290 superimposes a highlighting display by a marker at a position where the abnormal region is detected.

When the image synthesis portion 290 acquires diagnostic assistance information from the diagnostic assistance information generation portion 280, the image synthesis portion 290, for example, aligns and displays the captured image D and the diagnostic assistance image S2, as shown in FIG. 7.

### [Operation of Endoscopic System 500]

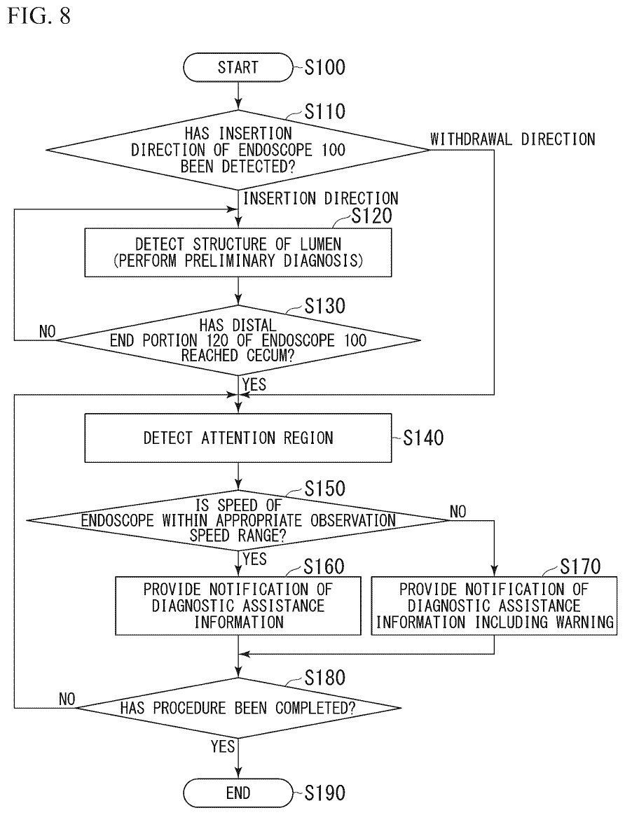

Next, the operation (diagnostic assistance method) of the endoscopic system 500 will be described. Specifically, a procedure of observing and treating the lumen wall of a hollow organ within the large intestine using the endoscopic system 500 will be described. Hereinafter, the description will be given along the flowchart of the endoscopic system 500 shown in FIG. 8.

### <Step S110>

In step S110, the endoscopic diagnosis assistance portion 230 detects an insertion direction of the endoscope 100. For example, the direction detection portion 260 acquires an output of the sensor 170 and detects an advancement/retraction direction (an insertion direction and a withdrawal direction) of the distal end portion 120 of the endoscope 100 based on the output of the sensor 170. Also, the speed detection portion 250 may detect the advancement/retraction direction of the endoscope 100 from a captured image D. When the insertion direction of the endoscope is the insertion direction, the endoscopic system 500 subsequently executes step S120. When the insertion direction of the endoscope is the withdrawal direction, the endoscopic system 500 subsequently executes step S140.

### <Step S120>

In step S120, the endoscopic diagnosis assistance portion 230 detects a structure of the large intestine. For example, the structure detection portion 241 detects the structure of the large intestine included in the captured image D from the captured image D. The endoscopic system 500 subsequently executes step S130.

In step S120, the endoscopic system 500 may also execute a preliminary diagnosis. The preliminary diagnosis refers to pre-detecting an abnormal region or a structural region to be carefully observed based on the captured image D captured when the endoscope 100 is inserted. A preliminary diagnosis result is used in step S140.

### <Step S130>

In step S130, the endoscopic diagnosis assistance portion 230 determines whether the distal end portion 120 of the endoscope 100 has reached the cecum. Specifically, the structure detection portion 241 determines whether the detected structure of the large intestine is the cecum. When the detected structure of the large intestine is not the cecum, the endoscopic system 500 continues step S120. When the detected structure of the large intestine is the cecum, the endoscopic system 500 executes step S140.

### <Step S140>

When the advancement/retraction direction of the endoscope 100 is the withdrawal direction or when the distal end portion 120 of the endoscope 100 has reached the cecum, observation and treatment in the large intestine are being performed by the practitioner. Accordingly, in step S140, the endoscopic diagnosis assistance portion 230 detects an attention region.

When a preliminary diagnosis has been performed in step S120 and an attention region has been pre-detected, the endoscopic diagnosis assistance portion 230 can detect the attention region in step S140 before the distal end portion 120 of the endoscope 100 actually passes through the attention region.

### <Step S150>

In step S150, the endoscopic diagnosis assistance portion 230 determines whether the speed of the endoscope is within the appropriate observation speed range. When the speed of the distal end portion 120 of the endoscope 100 is within the appropriate observation speed range, the endoscopic system 500 subsequently executes step S160. When the speed of the distal end portion 120 of the endoscope 100 is outside the appropriate observation speed range, the endoscopic system 500 subsequently executes step S170.

### <Step S160>

In step S160, the endoscopic diagnosis assistance portion 230 notifies the practitioner of diagnostic assistance information. The endoscopic system 500 subsequently executes step S180.

### <Step S170>

In step S170, the endoscopic diagnosis assistance portion 230 notifies the practitioner of diagnostic assistance information including a warning, thereby prompting the practitioner to carefully observe so that no abnormal region is overlooked. The endoscopic system 500 subsequently executes step S180.

### <Step S180>

In step S180, the endoscopic diagnosis assistance portion 230 determines whether the procedure has been completed. When the endoscopic diagnosis assistance portion 230 determines that the procedure has not been completed, step S140 and subsequent steps are executed. When the endoscopic diagnosis assistance portion 230 determines that the procedure has been completed, step S190 is executed and the control flow shown in FIG. 8 ends.

Also, in any of the steps of the control flow shown in FIG. 8, when an abnormal region is detected, the image synthesis portion 290, for example, superimposes an emphasized display with a marker at a position where the abnormal region is detected. The endoscopic diagnosis assistance portion 230 may also generate diagnostic assistance information for the abnormal region.

According to the endoscopic system 500 of the present embodiment, the endoscopic diagnosis assistance portion 230 (endoscopic diagnosis assistance device) can detect an attention region to be carefully observed and provide a notification to the practitioner so that an abnormal region such as a lesion is not overlooked and the region is carefully observed. As described above, in the present embodiment, the endoscopic image processing device 200 and the endoscopic image processing system 500 including: an image information acquisition portion 210 configured to acquire image information from the endoscope 100 having the imaging portion 150 configured to acquire an image of a lumen at the distal end portion 120 thereof; the lumen advancement/retraction speed detection portion 250 configured to detect the speed at which the distal end portion 120 of the endoscope 100 is advanceable into and retractable from (passes through) the lumen; and the appropriate observation speed determination portion 270 configured to determine whether or not a speed of the distal end portion 120 of the endoscope 100 in which the distal end portion 120 passes through a predetermined intraluminal attention region is within an appropriate observation speed range have been exemplified. The intraluminal attention region can be determined or inferred under predetermined conditions by detecting an acquisition position that is a position in the lumen where the distal end portion 120 of the endoscope 100 acquires an image. Naturally, these functions can be implemented by software. Specifically, these functions can be implemented by an endoscopic image processing program for causing a computer to execute: an image information acquisition step of acquiring image information from the endoscope 100 having the imaging portion 150 configured to acquire an image of a lumen at the distal end portion 120 thereof; a lumen advancement/retraction speed detection step of detecting a speed at which the distal end portion 120 of the endoscope 100 is advanceable into and retractable from the lumen; and an appropriate observation speed determination step of determining whether the speed of the distal end portion 120 passing through a specific intraluminal attention region is within the appropriate observation speed range.

Although the first embodiment of the present disclosure has been described above in detail with reference to the drawings, the specific configuration is not limited to this embodiment, and design modifications within the scope of the present disclosure are also included. Moreover, constituent elements shown in the above embodiment and the modified examples shown below can be configured in an appropriate combination.

### Second Embodiment

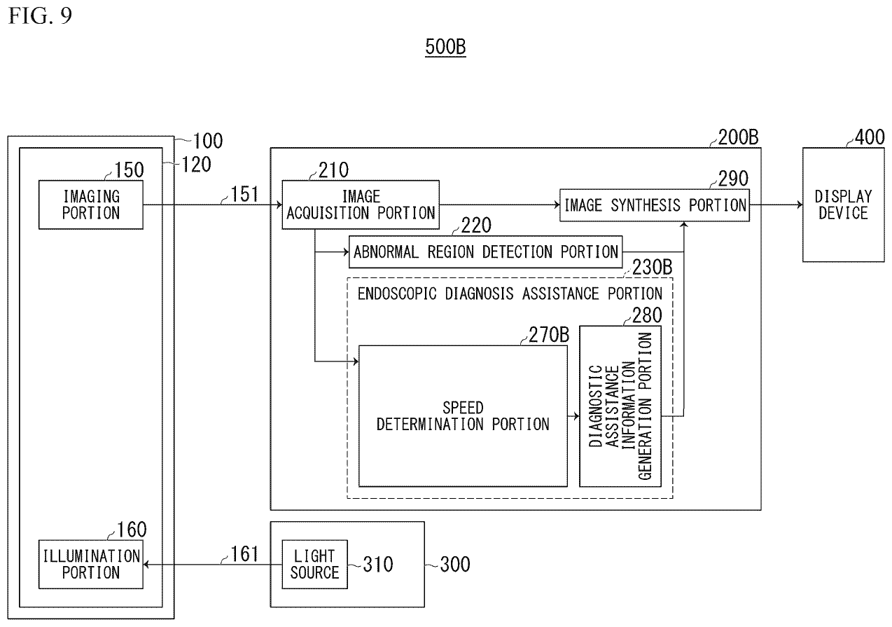

A second embodiment of the present disclosure will be described with reference to FIGS. 9 to 14. In the following description, constituent elements identical to those already described are denoted by the same reference signs, and redundant descriptions thereof will be omitted.

FIG. 9 is a functional block diagram of an endoscopic system 500B according to the second embodiment.

The endoscopic system 500B includes an endoscope 100, an image processing processor device 200B, a light source device 300, and a display device 400. As shown in FIG. 9, the image processing processor device 200B includes an image acquisition portion 210, an abnormal region detection portion 220, an endoscopic diagnosis assistance portion 230B, and an image synthesis portion 290.

The endoscopic diagnosis assistance portion 230B detects an attention region to be carefully observed from the captured image D and provides a notification to the practitioner so that the attention region is carefully observed. Moreover, the endoscopic diagnosis assistance portion 230B generates diagnostic assistance information for the attention region. The endoscopic diagnosis assistance portion 230B includes a speed determination portion 270B and a diagnostic assistance information generation portion 280.

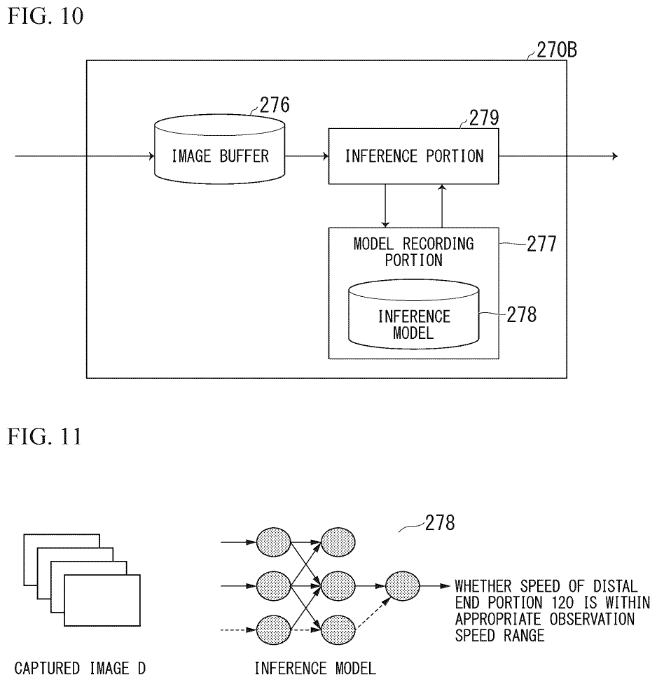

FIG. 10 is a functional block diagram of the speed determination portion 270B. The speed determination portion 270B detects an image feature of the attention region to be carefully observed from the captured image D of the lumen and determines whether the speed of the distal end portion 120 of the endoscope 100 passing through the attention region of the lumen is within an appropriate observation speed range. The speed determination portion 270B includes an image buffer 276, a model recording portion 277, and an inference portion 279.

The image buffer 276 is a part of the above-described recording portion, and is a nonvolatile recording medium. The image buffer 276 may also be a part of the above-described memory and may be a volatile recording medium. The image buffer 276 stores a plurality of transferred captured images D.

A plurality of captured images D (image frames) are recorded in the image buffer 276. When a recording capacity of the image buffer 276 is insufficient, the oldest captured image D is deleted. The plurality of captured images D recorded in the image buffer 276 may be captured images D of consecutive frames or may be captured images D in which a plurality of frames are thinned out from the consecutive frames.

The model recording portion 277 is a part of the above-described recording portion and is a nonvolatile recording medium. The model recording portion 277 records the inference model 278.

FIG. 11 is a conceptual diagram of the inference model 278.

The inference model 278 is a model obtained by machine learning using, as training data, image frames (captured images for learning) of endoscopes in a plurality of cases, and a result of determining whether the image frame is overlooked, determining a site that can be easily overlooked using the image frame, and annotating an examination speed suitable for examining the site. The inference model 278 is, for example, a neural network, and is trained through deep learning. Also, the inference model 278 is not limited to a neural network, and may be another machine learning model capable of outputting information for the input images.

The input of the inference model 278 is the captured image D, preferably a plurality of captured images (image frames) arranged in chronological order. The output of the inference model 278 is the determination of whether the speed of the distal end portion 120 of the endoscope 100 is within an appropriate observation speed range. The inference model 278 may also output the lumen structure included in the image frames and the optimal observation speed range.

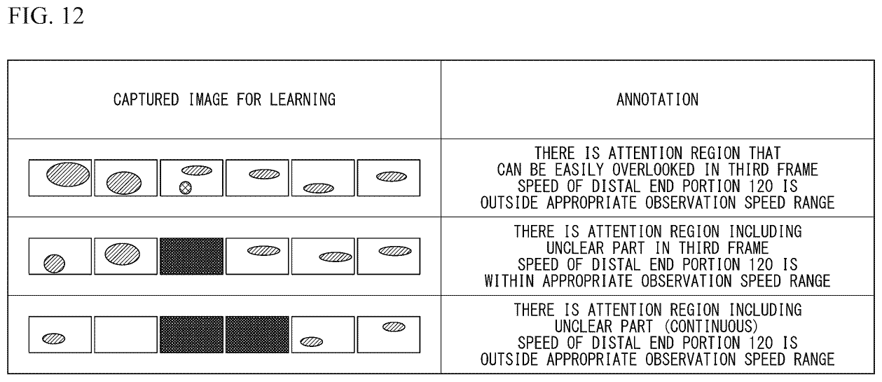

FIG. 12 is an explanatory diagram of training data.

As the training data, moving images (sequences of still images) obtained in endoscopic examinations of a plurality of cases are used. The training data is a combination of image frames (captured images for learning) and a result of determining whether the image frame is overlooked, determining a site that can be easily overlooked using the image frame, and annotating an examination speed suitable for examining the site. The inference model 278 is a model trained so that the annotation corresponding to the input image frame (captured image for learning) is output.

In the training data of FIG. 12, using the schemes of annotations such as “there is an attention region that can be easily overlooked in the third frame” and “there is an attention region including an unclear part in the third frame,” the inference model 278 can predict, based on an image frame three frames prior among image frames, that an attention region will appear three frames later, thereby prompting the user to pay attention in advance. Moreover, an image frame in which “there is an attention region including an unclear part (continuous)” is an example that can be easily selected as training data.

The inference model 278 determines whether suitable inference can be made according to whether abundant training data can be collected. The training data exemplified in FIG. 12 are advantageous in that appropriate images can be easily selected from an image group as training data using conventional lesion detection techniques or image degradation determination techniques.

In the training data of FIG. 12, examples in which unclear regions or easily-overlooked regions included in image frames are annotated as attention regions have been shown. However, the annotations included in the training data may be an annotation indicating that an image frame following an image frame with predetermined features is highly likely to include a lesion or may be an annotation indicating that it should not be overlooked because there is an image frame that is difficult to three-dimensionally reconstruct.” The greater the amount of training data, the higher the reliability of the trained inference model. Moreover, it is possible to implement a system equipped with an inference model configured to classify a plurality of image frames of moving images obtained in an endoscopic examination in a plurality of cases in chronological order and configured to be trained so that a result of determining a factor causing overlooking that has occurred in a second half of the plurality of image frames with respect to image frames classified as a first half of the image frames is annotated and used as training data and corresponding annotations are output with respect to the image frames that have been input. In this way, inference of what will happen later, such as “likelihood of overlooking” is enabled by the input of image frames obtained from the endoscope at an early stage in time, it is possible to detect in advance which regions are likely to be overlooked, and speedy determination is enabled.

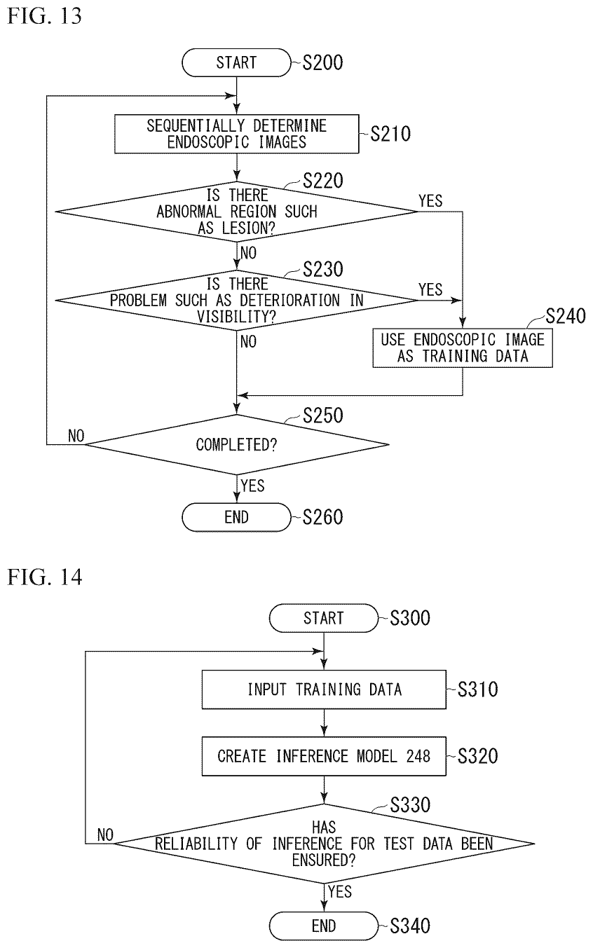

FIG. 13 is a flowchart showing the training data acquisition process.

In the training data acquisition process, in step S210, endoscopic images prepared for training are sequentially determined. When there is an abnormal region such as a lesion in the endoscopic image (step S220) or when there is a problem such as deterioration in visibility (step S230), the endoscopic image is acquired as training data. When the endoscopic image is acquired as training data, annotations indicating the presence of the abnormal region and the problem such as deterioration in visibility are also acquired. At this time, avoidance measures for the problem may also be recorded together as annotations.

FIG. 14 is a flowchart of a learning process of the inference model 278.

The inference model 278 is trained by a learning device. The learning device may be the image processing processor device 200 or may be an external computing device other than the image processing processor device 200.

In step S310, the above-described training data are input to the learning device. In step S320, the learning device creates the inference model 278 using the training data. In step S310, the learning device performs inference using the inference model 278 on test data (data similar to the training data but not used for training) to confirm whether reliability is ensured in the inference of the inference model 278. When the inference reliability of the inference model 278 is not ensured, the learning device performs step S310 again. At this time, at least a part of the training data is replaced to improve the inference model 278 into a more reliable one. With such measures, the endoscopic diagnosis assistance portion 230B can generate the inference model 278 based on intraluminal image information obtained in an intraluminal insertion process of endoscopic examinations in a plurality of cases, and output diagnostic assistance information for detecting specific target site images to be carefully observed during examination.

The inference portion 279 inputs the captured images D stored in the image buffer 276 to the inference model 278 and determines whether the speed of the distal end portion 120 of the endoscope 100 is within the appropriate observation speed range. The inference portion 279 outputs a determination result to the diagnostic assistance information generation portion 280.

Although the inference portion 279 may use conventional general-purpose arithmetic processing circuits such as a CPU and a field programmable gate array (FPGA), a graphics processing portion (GPU) or a tensor processing portion (TPU) specialized for matrix computation may be used because much of the processing of a neural network involves matrix multiplication. In recent years, artificial intelligence (AI)-dedicated hardware called a neural network processing portion (NPU) has been designed to be integrated and embedded with CPUs and other circuits, and may constitute a part of the processing circuit.

According to the endoscopic system 500B of the present embodiment, the endoscopic diagnosis assistance portion 230B (endoscopic diagnosis assistance device) can detect attention regions to be carefully observed and provides a notification to the practitioner so that the regions are carefully observed, thereby preventing the overlooking of abnormal regions such as lesions.

Although the second embodiment of the present disclosure has been described above in detail with reference to the drawings, the specific configuration is not limited to this embodiment, and design modifications within the scope of the present disclosure are also included. Moreover, constituent elements shown in the above embodiment and the modified examples shown below can be configured in an appropriate combination.

In the above-described embodiment, the endoscopic diagnosis assistance portion (endoscopic diagnosis assistance device) provides diagnostic assistance for medical endoscopic images. However, a diagnostic target of the endoscopic diagnosis assistance portion (endoscopic diagnosis assistance device) is not limited to medical endoscopic images. The endoscopic diagnosis assistance portion (endoscopic diagnosis assistance device) may provide diagnostic assistance for captured images acquired from other imaging devices of mobile devices such as cameras, video cameras, industrial endoscopes, microscopes, robots having image acquisition functions, smartphones, mobile phones, smartwatches, tablet terminals, notebook PCs, and the like.

As described above, in one embodiment, an endoscopic image processing system includes: an image information acquisition portion configured to detect an acquisition information that is a position within a lumen whose image is acquired by an imaging portion provided at a distal end portion of the endoscope; a lumen passage speed detection portion configured to detect a speed at which the distal end portion of the endoscope is advanced into or retracted from the lumen; and an appropriate observation speed determination portion configured to determine, based on an inference mode, whether or not the speed of the distal end portion of the endoscope passing through an attention region capable of being determined under a predetermined condition is within a predetermined speed range for the attention region relative to the detected acquisition position, wherein the inference model was obtained by machine learning using: previously captured image frames captured in multiple cases; and a result of determining the attention region for the previously captured image frames to annotate an examination speed appropriate for examining the attention region, as training data. Each “portion” can be performed/implemented by a different computer/processor.

The present disclosure can be applied to an endoscopic system and the like.

What is claimed is:

1. An endoscopic diagnosis assistance method comprising:
detecting an acquisition position that is a position within a lumen whose image is acquired by an imaging portion provided at a distal end portion of an endoscope;
detecting a speed at which the distal end portion of the endoscope is advanced into or retracted from the lumen; and
determining, based on an inference model, whether or not the speed of the distal end portion of the endoscope passing through an attention region capable of being determined under a predetermined condition is within a predetermined speed range for the attention region relative to the detected acquisition position, wherein the inference model was obtained by machine learning using:
previously captured image frames captured in multiple cases; and
a result of determining the attention region for the previously captured image frames to annotate an examination speed appropriate for examining the attention region,
as training data.

2. The endoscopic diagnosis assistance method according to claim 1, wherein detecting the speed of the distal end portion comprises detecting the speed of the distal end portion based on movement of a physical object between image frames captured by the endoscope.

3. The endoscopic diagnosis assistance method according to claim 1, wherein detecting the speed of the distal end portion comprises detecting the speed of the distal end portion based on an output of a sensor provided at the distal end portion of the endoscope.

4. The endoscopic diagnosis assistance method according to claim 1, wherein the acquisition position is detected based on the acquired image.

5. The endoscopic diagnosis assistance method according to claim 4, wherein the acquisition position is inferred based on an inference model trained with training data in which annotations have been added to sites included in previously recorded images for inferring sites of the lumen from the image.

6. The endoscopic diagnosis assistance method according to claim 1,
wherein a magnetic coil is stored in the distal end portion, and
wherein the acquisition position is detected based on magnetism received from the magnetic coil.

7. The endoscopic diagnosis assistance method according to claim 1, further comprising:
determining whether the endoscope is being advanced into or retracted from the lumen;
in response to determining that the endoscope is being retracted from the lumen, determining whether or not the speed of the distal end portion of the endoscope passing through the attention region is within the predetermined speed range.

8. The endoscopic diagnosis assistance method according to claim 1, further comprising:
determining whether the endoscope is being advanced into or retracted from the lumen; and
detecting the attention region based on an image acquired by the endoscope when the endoscope was inserted into the lumen before the endoscope is retracted from the lumen.

9. The endoscopic diagnosis assistance method according to claim 7, further comprising:
detecting sites of the lumen from images acquired by the endoscope,
wherein determining whether the endoscope is being advanced into or retracted from the lumen comprises determining whether the endoscope is being advanced into or retracted from the lumen based on a history of the sites of the lumen detected from the images acquired by the endoscope.

10. The endoscopic diagnosis assistance method according to claim 7, wherein determining whether the endoscope is being advanced into or retracted from the lumen comprises determining whether the endoscope is being advanced into or retracted from the lumen based on an output of a sensor provided at the distal end portion of the endoscope.

11. The endoscopic diagnosis assistance method according to claim 1, further comprising:
detecting at least one of a lesion and a structure segmented in the lumen as the attention region.

12. The endoscopic diagnosis assistance method according to claim 1, further comprising:
in response to determining that the speed of the distal end portion of the endoscope is not within the predetermined speed range, generating diagnosis assistance information including a warning for the attention region.

13. The endoscopic diagnosis assistance method according to claim 12, further comprising:
controlling a display device to display the diagnosis assistance information including the warning is displayed on a display device, and
wherein the displayed diagnosis assistance information includes at least one of:
a position of the distal end portion in the lumen;
information indicating the warning; and
speed information obtained by visualizing the speed of the distal end portion and the predetermined speed range.

14. An endoscopic diagnosis assistance method comprising;
continuously acquiring image frames of a lumen from an endoscope;
detecting an image feature of an attention region to be observed from the image frames of the lumen;
determining whether or not a speed of the endoscope passing through an attention region of the lumen is within a predetermined speed range; and
generating diagnosis assistance information including a warning for the attention region when the speed of the endoscope is outside the predetermined speed range.

15. The endoscopic diagnosis assistance method according to claim 14, wherein determining whether or not the speed of the endoscope is within the predetermined speed range comprises determining whether or not the speed of the endoscope is within the predetermined speed range based on an inference model obtained according to machine learning using:
image frames acquired in a plurality of cases; and
a result of determining whether the image frames are overlooked, determining a site that can be overlooked using the image frame, and annotating an examination speed suitable for examining the site.

16. The endoscopic diagnosis assistance method according to claim 14, wherein generating the diagnostic assistance information comprises generating the diagnostic informing in response to determining that the speed of the endoscope is outside the predetermined speed range.

17. A non-transitory computer-readable storage medium storing an inference model, wherein the inference model, when applied by a computer, is configured to:
classify a plurality of image frames of moving images obtained in an endoscopic examination in a plurality of cases in chronological order; and
be trained so that a result of determining a factor causing overlooking that has occurred in a second half of the plurality of image frames with respect to image frames classified as a first half of the image frames is annotated and used as training data and corresponding annotations are output with respect to the image frames that have been input, based on an inference model obtained by machine learning using:
image frames of the endoscope in multiple cases; and
a result of determining an attention region for the image frames to annotate an examination speed appropriate for examining the attention region, as training data.

18. An endoscopic image processing device comprising:
at least one processor configured to:
detect an acquisition position that is a position within a lumen whose image is acquired by an imaging portion provided at a distal end portion of the endoscope;
detect a speed at which the distal end portion of the endoscope is advanced into or retracted from the lumen; and
determine, based on an inference mode, whether or not the speed of the distal end portion of the endoscope passing through an attention region capable of being determined under a predetermined condition is within a predetermined speed range for the attention region relative to the detected acquisition position, wherein the inference model was obtained by machine learning using:
previously captured image frames captured in multiple cases; and
a result of determining the attention region for the previously captured image frames to annotate an examination speed appropriate for examining the attention region,
as training data.

19. An endoscopic image processing system comprising:
an image information acquisition portion configured to detect an acquisition information that is a position within a lumen whose image is acquired by an imaging portion provided at a distal end portion of an endoscope;
a lumen passage speed detection portion configured to detect a speed at which the distal end portion of the endoscope is advanced into or retracted from the lumen; and
an appropriate observation speed determination portion configured to determine, based on an inference mode, whether or not the speed of the distal end portion of the endoscope passing through an attention region capable of being determined under a predetermined condition is within a predetermined speed range for the attention region relative to the detected acquisition position, wherein the inference model was obtained by machine learning using:
previously captured image frames captured in multiple cases; and
a result of determining the attention region for the previously captured image frames to annotate an examination speed appropriate for examining the attention region,
as training data.

20. A non-transitory computer-readable storage medium storing a program for causing a computer to execute:
detecting an acquisition position that is a position within a lumen whose image is acquired by an imaging portion provided at a distal end portion of the endoscope;
detecting a speed at which the distal end portion of the endoscope is advanced into or retracted from the lumen; and
determining, based on an inference mode, whether or not the speed of the distal end portion of the endoscope passing through an attention region capable of being determined under a predetermined condition is within a predetermined speed range for the attention region relative to the detected acquisition position, wherein the inference model was obtained by machine learning using:
previously captured image frames captured in multiple cases; and
a result of determining the attention region for the previously captured 5 image frames to annotate an examination speed appropriate for examining the attention region,
as training data.
